(19) United States
(12) Patent Application Publication
(10) Pub. No.: US 2024/0199693 A1
(43) Pub. Date: Jun. 20, 2024

(54) HEPTAPEPTIDE AMIDES FOR USE IN THE TREATMENT OF HMGB1-DEPENDENT DISEASES

(71) Applicant: FEDERAL STATE BUDGETARY INSTITUTION SCIENTIFIC CENTER OF BIOMEDICAL TECHNOLOGIES OF FEDERAL MEDICAL, Moscow (RU)

(72) Inventors: Vladislav Nikolaevich KARKISCHENKO, Moscow (RU); Veronika Igorevna SKVORTSOVA, Moscow (RU); Igor Anatolievich POMYTKIN, Moscow (RU); Nikolai Nikolaevich KARKISCHENKO, Moscow (RU)

(21) Appl. No.: 18/287,512

(22) PCT Filed: Apr. 14, 2022
(86) PCT No.: PCT/RU2022/000112

§ 371 Date: Oct. 19, 2023

(30) Foreign Application Priority Data
Apr. 22, 2021 (RU) 2021111474

(51) Int. Cl. C07K 7/06; A61K 38/08; A61P 19/02

(52) U.S. Cl. CPC C07K 7/06; A61K 38/08; A61P 19/02

(57) ABSTRACT

The present invention relates to a compound of formula (I): where Z is (II) in which R¹ and R² are identical or different and are hydrogen or a lower alkyl chain having up to 4 carbon atoms or a pharmaceutically acceptable salt thereof. Further, the present invention relates to pharmaceutical compositions or dependent inflammatory or autoimmune disease selected from the group consisting of rheumatoid arthritis, osteoarthritis, ankylosing spondylitis, periodontitis, allergic rhinitis, chronic rhinosinusitis without nasal polyps, chronic rhinosinusitis with nasal polyps, pneumonia, chronic obstructive pulmonary disease, asthma, acute adult respiratory syndrome, diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, diabetic foot ulceration, colitis, inflammatory bowel disease, and ocular disease. Preferably, the pharmaceutically acceptable salt is acetate.

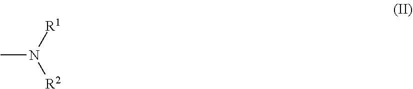

### FIELD OF THE INVENTION

The present invention relates to heptapeptide amides for use in medicine, particularly for the treatment of high mobility group box-1 (HMGB1) dependent inflammatory and autoimmune diseases.

### BACKGROUND OF THE INVENTION

High mobility group box-1 (HMGB1) is a nuclear protein, also known as amphoterin and HMG-1, that normally possesses numerous biological activities. Upon secretion from cells to extracellular space, HMGB1 can bind to multiple receptors including the receptor for advanced glycation end-products (RAGE) and toll-like receptors (TLR2, TLR4, and etc.), thereby mediating inflammatory and immune responses. Kang et al. Mol. Aspects Med. 2014, 40: 1-116. Yang H et al. Front Immunol. 2020, 11:484. The extracellular HMGB1 is implicated in a wide variety of inflammatory autoimmune diseases, as described in Harris H E, et al. Nat

Rev Rheumatol. 2012, 8:195-202, and other references cited below. HMGB1 has been identified as a mediator of endotoxin lethality and target in the treatment of sepsis. Wang H, et al. Science. 1999, 285(5425):248-51. HMGB1 has been identified as a pathogenic factor in numerous inflammatory lung diseases, such as pulmonary tuberculosis, chronic obstructive pulmonary disease (COPD), pulmonary fibrosis, asthma, acute respiratory distress-syndrome (ARDS), pneumonia, severe pulmonary inflammation, and cytokine storm caused by influenza virus and COVID-19. Kudo D, et al. Clin Exp Immunol. 2013, 173(2):276-87. Ding J, et al. J Cell Mol Med. 2017, 21(6):1046-1057. Hou C, et al. Mol Med. 2011, 17:807-815. Andersson U, et al. Mol Med. 2020, 26(1):42. HMGB1 has been shown to be implicated in upper airway inflammatory diseases such as rhino-sinusal inflammation, allergic rhinitis, chronic rhinosinusitis with or without nasal polyps and targeting HMGB1 has shown to be useful for mitigation symptoms of upper airway inflammatory diseases. Bellussi L M, et al. Int Arch Otorhinolaryngol. 2017, 21(4):390-398. HMGB1 has been identified as a pathogenic factor and target for the treatment of osteoarthritis, a degenerative joint disorder affecting over 250 million people worldwide. Feng Y, et al. Cell Mol Med. 2018, 22(2):1283-1291. HMGB1 is implicated in the development of diabetic complications, such as diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, and diabetic foot ulceration. Biscetti F, et al. Int J Mol Sci. 2019, 20(24):6258. Wu H, et al. Mediators Inflamm. 2016, 2016:3896147. Hafez Y M, et al. Diabetes Metab Syndr. 2018, 12(6):1065-1070. HMGB1 has been identified as a pathogenic factor and target for the treatment of rheumatoid arthritis, an autoimmune disease occurring in as much as 1-2% of the population. Kokkola R, et al. Arthritis Rheum. 2002, 46(10):2598-603. Taniguchi N, et al. Arthritis Rheum. 2003, 48(4):971-81. Andersson U, Erlandsson-Harris H. J Intern Med. 2004, 255(3):344-50. Pullerits R, et al. Arthritis Rheum. 2003, 48(6):1693-700. Kokkola R, et al. Arthritis Rheum. 2003, 48(7):2052-8. Sundberg E, et al. Arthritis Res Ther. 2008, 10(2):R33. HMGB1 is implicated in periodontitis, an infectious disease which causes inflammation in gingiva and other periodontal tissues and affects more than 60% of the population over the world. Shaddox L M, Walker C B. Clin Cosmet Investig Dent. 2010; 2:79-91. Sun Y, et al. Dose Response. 2020, 18(3):1559325820952660. Yamashiro K, et al. Front lmmunol. 2020, 11:1461. HMGB1 is implicated in the gut mucosal inflammation and, onset and progression of gastrointestinal diseases Sappington P L, et al. Gastroenterology. 2002, 123(3):790-802. Dai S, et al. J Biol Chem 2010, 285(7):4995-5002. Vitali R, et al. PLoS One 2013, 8(6):e66527. HMGB1 is implicated in the autoimmune diseases, such as antineutrophil cytoplasmic antibody (ANCA)-associated vasculitis, Behcet disease, systemic lupus erythematosus, rheumatoid arthritis, ankylosing spondylitis, Sjogren syndrome and myositis, thereby representing the target in the treatment thereof. Harris H E, et al. Nat Rev Rheumatol. 2012, 8:195-202. Magna M, Pisetsky D S. Mol Med. 2014, 20(1):138-46. Zhu B, et al. Medicine (Baltimore). 2018, 97(29):e11531. HMGB1 is implicated in ocular diseases such as uveitis, Behcet's disease, age-related macular degeneration, glaucoma, and keratitis. Liu Y et al. J Ophthalmol. 2018, 2018:5195290. Thus, there is a great need in safe and effective agents blocking activities of extracellular HMGB1 or protecting against HMGB1 release to the extracellular environment for the treatment or prevention of HMGB1 dependent inflammatory and autoimmune diseases.

Several pharmacological approaches targeting extracellular HMGB1 have been demonstrated in preclinical studies, including the use of HMGB1 monoclonal antibodies, resveratrol, α2-adrenoceptor agonist dexmedetomidine, soluble thrombin receptor thrombomodulin, glycyrrhizic acid, and acute phase protein haptoglobin. However, there is currently no commercially available effective anti-HMGB1 agent for treating HMGB1 dependent inflammatory and autoimmune disorders. Yang H et al. Front Immunol. 2020, 11:484.

The modified [D-Ala²]-dynorphin (1-7), having the formula H-Tyr-D-Ala-Gly-Phe-Leu-Arg-Arg-OH, had been described by Chavkin C et al. PNAS, 78(10), 6543-7, 1981, as a compound obtained by enzymatic cleavage of [D-Ala²]-dynorphin (1-13)-NH₂ with trypsin. However, [D-Ala²]-dynorphin amide (1-7), having the formula H-Tyr-D-Ala-Gly-Phe-Leu-Arg-Arg-NH₂ is unknown from the prior art, including the Chavkin C et al. publication.

U.S. Pat. No. 4,707,468 discloses a polypeptide defined by the below shown general formula for use as an analgesic

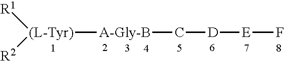

wherein R¹ and R² can be the same or different and each is hydrogen, lower alkyl or lower alkenyl; A is selected from the group consisting of D-Met, D-Ala, D-Ser, D-Cys, D-Thr, Gly and Sar, provided that when A is D-Cys, it is bonded with L-Cys or D-Cys located in position 5 through a S—S bond to effect intramolecular ring closure; B is L-Phe or D-Phe in which the benzene ring can be substituted or an α-N-alkyl derivative thereof; C is selected from the group consisting of L-Leu, L-Ile, L-Nle, L-tert.-Leu, L-Met, L-Met(0), L-Ser, L-Cys, L-Val, D-Cys and α-N-alkyl derivatives thereof, provided that when A is D-Cys, C is L-Cys or D-Cys and C is bonded with A through said S—S bond; D and E each is selected from the group consisting of L-Arg, D-Arg, L-Lys, D-Lys, L-homo-Arg, D-homo-Arg, L-Orn, D-Orn and α-N-alkyl derivatives thereof; F is OR³,

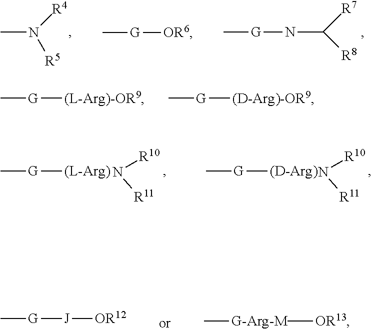

in which R³, R⁴, R⁵ R⁶, R⁷, R⁸, R⁹, R¹⁰, R¹¹, R¹², and R¹³ are the same or different and each is hydrogen or lower alkyl, G is selected from the group consisting of Gly, Ala, Val, Leu, Ile, Ser, Thr, Cys, Met, Asp, Glu, Asn, Gln, Pro, Lys, Orn, Arg, His, Phe, Tyr, Trp, tert.-Leu, 2-aminoisobutyric acid, α-methyl-Leu, β-alanine, γ-aminobutyric acid and α-N-alkyl derivatives thereof, J is selected from the group consisting of Gly, Sar, L-Ala, D-Ala, L-Phe, D-Phe, L-Asp and D-Asp, and M is selected from the group consisting of D-Pro, D-ala and D-Glu, provided that at least one of A, B, C, D, E and F comprises a D-amino acid or an N-alkyl derivative of a D-amino acid or a L-amino acid, or a pharmacologically acceptable salt of said polypeptide.

SU 1433415A3 discloses a method of synthesis of a peptide for use an analgesic, defined by general formula

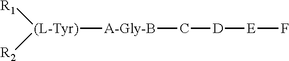

wherein R₁=R₂-hydrogen or one of R₁ and R₂ is hydrogen, other is C₁-C₄-alkyl; A-Gly D-Cys, bound with C, if C-Cys; B-Phe, (n-NO₂)Phe; C-Leu, Met(O), Cys, bound with A, if A-D-Cys; D-Arg, CH₃Arg, Lys; E-Arg, CH₃Arg; F—NH₂, D-Leu-X, wherein X—OH, NH₂, OC₂H₅, NHC₂H₅ or D-Leu-Arg-X, wherein X—NH₂, NHC₂H₅, by the stepwise increasing the peptide chain, starting from BOC-protected C-terminal amino acid using the carbodiimide method for the activation of carboxylic groups and the mixed anhydrides method, and removing the protected groups from the obtained protected peptide.

Although patents U.S. Pat. No. 4,707,468 and SU 1433415A3 have disclosed the peptide of generic formula for use as an analgesic

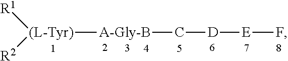

a compound of formula H-Tyr-D-Ala-Gly-Phe-Leu-Arg-Arg-NH₂ and alkyl derivatives thereof at C-terminal amido-group are novel, since they have not been explicitly mentioned in U.S. Pat. No. 4,707,468 and SU 1433415A3. Moreover, in order to arrive at the specific amino acid sequence of the compound of formula H-Tyr-D-Ala-Gly-Phe-Leu-Arg-Arg-NH₂ and the alkyl derivatives thereof, a selection has to be made from several lists of substituents indicated as 1 through 8 in the above generic formula, with R¹=R²=H being selected from the list 1, A=D-Ala being selected from the list 2, B=Phe being selected from the list 4, C=Leu being selected from the list 5, D=Arg being selected from the list 6, E=Arg being selected from the list 7, and F=N H2 being selected from the list 8.

Surprisingly, we found that administering the compound of formula H-Tyr-D-Ala-Gly-Phe-Leu-Arg-Arg-NH₂ or the alkyl derivatives at C-terminal amido-group thereof reduces pathologically elevated levels of extracellular HMGB1 in mammals. Therefore, these compounds can be used for the prevention and treatment of HMGB1-dependent inflammatory and autoimmune diseases. This effect is totally unexpected over U.S. Pat. No. 4,707,468 and SU 1433415A3, since the anti-HMGB1 effect discovered in the present invention is unrelated in any way to analgesia indicated in U.S. Pat. No. 4,707,468 and SU 1433415A3.

### SUMMARY OF THE INVENTION

The present invention relates to a compound of formula (I):

H-Tyr-D-Ala-Gly-Phe-Leu-Arg-Arg-Z (I)

where Z is

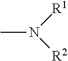

in which R¹ and R² are identical or different and are hydrogen or a lower alkyl chain having up to 4 carbon atoms or a pharmaceutically acceptable salt thereof for use in the treatment of HMGB1-dependent inflammatory and autoimmune diseases.

A first aspect of the invention relates to a compound of formula (I):

H-Tyr-D-Ala-Gly-Phe-Leu-Arg-Arg-Z (I)

where Z is

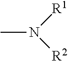

in which R¹ and R² are identical or different and are hydrogen or a lower alkyl chain having up to 4 carbon atoms or a pharmaceutically acceptable salt thereof.

According to another aspect, the invention relates to a pharmaceutical composition comprising an effective amount of a compound of formula (I):

H-Tyr-D-Ala-Gly-Phe-Leu-Arg-Arg-Z (I)

where Z is

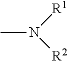

in which R¹ and R² are identical or different and are hydrogen or a lower alkyl chain having up to 4 carbon atoms or a pharmaceutically acceptable salt thereof and a pharmaceutically acceptable excipient.

According to another aspect, the invention relates to a compound of formula (I):

H-Tyr-D-Ala-Gly-Phe-Leu-Arg-Arg-Z (I)

where Z is

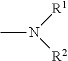

in which R¹ and R² are identical or different and are hydrogen or a lower alkyl chain having up to 4 carbon atoms or a pharmaceutically acceptable salt thereof for use as a medicament.

According to another aspect, the invention relates to a compound of formula (I):

H-Tyr-D-Ala-Gly-Phe-Leu-Arg-Arg-Z (I)

where Z is

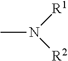

in which R¹ and R² are identical or different and are hydrogen or a lower alkyl chain having up to 4 carbon atoms or a pharmaceutically acceptable salt thereof for use in the treatment of a HMGB1 dependent inflammatory or autoimmune disease selected from the group consisting of rheumatoid arthritis, osteoarthritis, ankylosing spondylitis, periodontitis, allergic rhinitis, chronic rhinosinusitis without nasal polyps, chronic rhinosinusitis with nasal polyps, pneumonia, chronic obstructive pulmonary disease, asthma, acute adult respiratory syndrome, diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, diabetic foot ulceration, colitis, inflammatory bowel disease, and ocular disease.

In a preferred embodiment, the compound (I) is selected from the group consisting of H-Tyr-D-Ala-Gly-Phe-Leu-Arg-Arg-NH₂, H-Tyr-D-Ala-Gly-Phe-Leu-Arg-Arg-NHC₂H₅, H-Tyr-D-Ala-Gly-Phe-Leu-Arg-Arg-NHC₄H₉, and H-Tyr-D-Ala-Gly-Phe-Leu-Arg-Arg-N(CH₃)₂.

In a more preferred embodiment, the compound (I) is H-Tyr-D-Ala-Gly-Phe-Leu-Arg-Arg-NH₂.

In a preferred embodiment, the pharmaceutically acceptable salt of the compound (I) is acetate.

### DETAILED DESCRIPTION OF THE INVENTION

All terms and definitions explained throughout the text of specification of the invention relate to all aspects and embodiments of the invention, unless otherwise specified.

The present invention relates to a compound of formula (I):

H-Tyr-D-Ala-Gly-Phe-Leu-Arg-Arg-Z (I)

where Z is

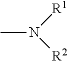

in which R¹ and R² are identical or different and are hydrogen or a lower alkyl chain having up to 4 carbon atoms or a pharmaceutically acceptable salt thereof for use in the treatment of a HMGB1-dependent inflammatory or autoimmune disease.

As used herein, the term “HMGB1” refers to a high mobility group box-1 protein (also known as high mobility group protein B1, amphoterin, and HMG-1) as well as to any and all post-translational modifications thereof, including without limitation acetylated HMGB1, phosphorylated HMGB1, ADP-ribosylated HMGB1, methylated HMGB1, glycosylated HMGB1, partially or fully reduced HMGB1, partially or fully oxidized HMGB1, all-thiol HMGB1, and disulfide HMGB1. HMGB1 used herein includes HMGB1 from any species, including mammalian HMGB1s, such as human HMGB1, rodent HMGB1, bovine HMGB1, porcine HMGB1, and their amino acid sequences disclosed in Gen Bank and UniProtKB/Swiss-Prot databases, for instance, UniProtKB/Swiss-Prot Accession No. P09429 for human HMGB1.

As used herein, the term “HMGB1-dependent an inflammatory or autoimmune disease” refers to an inflammatory or autoimmune disease associated with HMGB1 activity, HMGB1 overexpression, HMGB1 overproduction, and/or increase in extracellular HMGB1 levels.

A first aspect of the invention relates to a compound of formula (I):

H-Tyr-D-Ala-Gly-Phe-Leu-Arg-Arg-Z (I)

where Z is

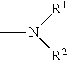

in which R¹ and R² are identical or different and are hydrogen or a lower alkyl chain having up to 4 carbon atoms or a pharmaceutically acceptable salt thereof.

In a preferred embodiment, the compound (I) is selected from the group consisting of H-Tyr-D-Ala-Gly-Phe-Leu-Arg-Arg-NH₂, H-Tyr-D-Ala-Gly-Phe-Leu-Arg-Arg-NHC₂H₅, H-Tyr-D-Ala-Gly-Phe-Leu-Arg-Arg-NHC₄H₉, and H-Tyr-D-Ala-Gly-Phe-Leu-Arg-Arg-N(CH₃)₂ acetate.

In a more preferred embodiment, the compound (I) is H-Tyr-D-Ala-Gly-Phe-Leu-Arg-Arg-NH₂ acetate, the name L-tyrosyl-D-alanyl-L-glycyl-L-phenylala nyl-L-leucyl-L-arginyl-L-arginine amide acetate.

The term “pharmaceutically acceptable” refers to molecular entities and compositions that are of sufficient purity and quality for use in the formulation of a composition of the present invention and that, when appropriately administered to a mammal, e.g., to a human, do not produce a toxic, adverse, allergic or other untoward reaction.

The present invention relates to all pharmaceutically acceptable salts of the compound (I). Examples of such pharmaceutically acceptable salts include, but are not limited to, chloride, bromide, sulfate, acetate, pyruvate, malate, fumarate, and citrate.

The compound (I) can be obtained by the solid-phase synthesis or by any other method of peptide synthesis known from the art, as described, for instance, in Benton L. Chemistry of peptide synthesis. Taylor&Francis, 2006.

For illustrative purposes, the reaction schemes depicted below provide potential routes for synthesizing the compound (I) as well as key intermediates. For a more detailed description of the individual reaction steps, see the Examples section below. Those skilled in the art will appreciate that other synthetic routes, for instance, those indicated in SU 1433415A3, may be used to synthesize the compounds. Although specific starting materials and reagents are depicted in the schemes and discussed below, other starting materials and reagents can be easily substituted to provide a variety of derivatives and/or reaction conditions.

A general process of synthesis of the compound (I) includes a reaction of N-protected, e.g., with Cbz-group, methyl ester (II) with a primary or secondary amine HNR¹R² in appropriate solvent, e.g., methanol; and subsequent deprotecting the compound (III) by hydrogenolysis in the presence of catalyst, e.g., palladium on charcoal or Raney nickel.

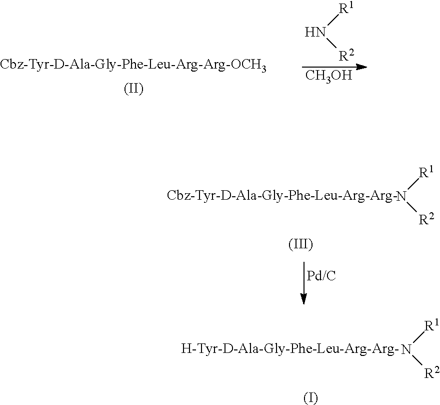

A process of synthesis of the N-protected, e.g., with Cbz-group, heptapeptide methyl ester (II) includes steps as follows: protecting hexapeptide (IV) at N-terminus with benzyloxycarbonyl-group (Cbz-); and reacting resulting compound (V) with methyl ester of L-arginine in the presence of dehydrating agent, e.g., dicyclohexylcarbodiimide (DCC).

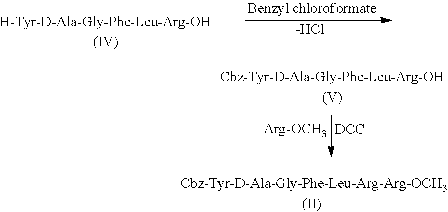

According to another aspect, the invention relates to a pharmaceutical composition for treatment of a HMGB1-dependent inflammatory or autoimmune disease comprising a compound of formula (I):

H-Tyr-D-Ala-Gly-Phe-Leu-Arg-Arg-Z (I)

where Z is

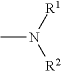

in which R¹ and R² are identical or different and are hydrogen or a lower alkyl chain having up to 4 carbon atoms or a pharmaceutically acceptable salt thereof and a pharmaceutically acceptable excipient.

In practicing the invention, the pharmaceutical composition may be administered by any conventional route known from the art, e.g., intravenously, intramuscularly, intraperitoneally, subcutaneously, intraarticularly, transdermally, rectally, topically, intranasally, by inhalation, and orally in a variety of dosage forms, including without limitation injections, infusions, lotions, ointments, gels, creams, suspensions, emulsions, suppositories, drops, patches, aerosols, or sprays.

As used herein, the term “pharmaceutically acceptable excipient” refers to a substance that is not a therapeutic agent and includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Non-exclusive examples of excipients suitable for use in liquid dosage forms include purified water, water for injection, buffer systems to maintain a pH of 3.0-8.5, regulators of tonicity of solutions, antimicrobial preservatives, surfactants such as no-ionic and amphiphilic surfactants, stabilizing agents, and emulsifiers. Non-exclusive examples of excipients suitable for use in solid dosage forms include starches, sugars, micro-crystalline cellulose, diluents, granulating agents, lubricants, binders, and disintegrating agents, and suppository bases such as cocoa butter, natural and synthetic triglycerides, polyethylene glycol polymers, glycerinated gelatin, and cocoa butter substitutes such as Witepsol.

In practicing the invention, the compositions may contain the compound (I) or a pharmaceutically acceptable salt thereof in an amount from 0.001 to 99.999%.

In practicing the present invention, the composition of the invention can be prepared by procedures well-known from the art. Such procedures include, but are not limited to, mixing compound (I) or a pharmaceutically acceptable salt thereof with other ingredients of the composition in conventional manner. Guidance for the preparation of compositions of the invention can be found in “Remington: The science and practice of pharmacy” 20th ed. Mack Publishing, Easton PA, 2000 ISBN 0-912734-04-3 and “Encyclopaedia of Pharmaceutical Technology”, edited by Swarbrick, J. & J. C. Boylan, Marcel Dekker, Inc., New York, 1988 ISBN 0-8247-2800-9 or a newer edition.

According to another aspect, the invention relates to a compound of formula (I):

H-Tyr-D-Ala-Gly-Phe-Leu-Arg-Arg-Z (I)

where Z is

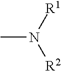

in which R¹ and R² are identical or different and are hydrogen or a lower alkyl chain having up to 4 carbon atoms or a pharmaceutically acceptable salt thereof for use as a medicament.

As used herein, the term “medicament” refers to a product for use in treating, preventing or ameliorating a chronic or acute disease, disorder or condition.

In practicing the invention, the medicament may be a lyophilized powder of compound (I) or a pharmaceutical salt thereof or a pharmaceutical composition comprising compound (I) or a pharmaceutical salt thereof and a pharmaceutically acceptable excipient.

According to another aspect, the invention relates to a compound of formula (I):

H-Tyr-D-Ala-Gly-Phe-Leu-Arg-Arg-Z (I)

where Z is

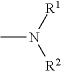

in which R¹ and R² are identical or different and are hydrogen or a lower alkyl chain having up to 4 carbon atoms or a pharmaceutically acceptable salt thereof for use in the treatment of a HMGB1 dependent inflammatory or autoimmune disease selected from the group consisting of rheumatoid arthritis, osteoarthritis, ankylosing spondylitis, periodontitis, allergic rhinitis, chronic rhinosinusitis without nasal polyps, chronic rhinosinusitis with nasal polyps, pneumonia, chronic obstructive pulmonary disease, asthma, acute adult respiratory syndrome, diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, diabetic foot ulceration, colitis, inflammatory bowel disease, and ocular disease.

As used herein, the term “treatment” refers to inhibition or prevention of the progression of a disease, disorder, or condition, or one or more symptoms thereof. The term “treatment” refers to both therapeutic treatment and prophylactic or preventative measures. Those in need of treatment include those already with the disorder as well as those prone to having the disorder or diagnosed with the disorder or those in which the disorder is to be prevented.

Further, the invention provides a method of the treatment of a HMGB1-dependent inflammatory or autoimmune disease comprising administering an effective amount of a compound of formula (I):

H-Tyr-D-Ala-Gly-Phe-Leu-Arg-Arg-Z (I)

where Z is

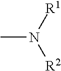

in which R¹ and R² are identical or different and are hydrogen or a lower alkyl chain having up to 4 carbon atoms or a pharmaceutically acceptable salt thereof to a subject in need thereof.

As used herein, the term “subject” refers to a mammal. Non-exclusive examples of

such mammals include primates (e.g., humans, male or female), cows, sheep, goats, pigs, horses, dogs, cats, rabbits, rats, mice, and the like. In a preferred embodiment, the subject is a human.

As used herein, a subject is “in need of” a treatment if such subject would have a benefit medically, physiologically, or in quality of life from such treatment.

The term “a therapeutically effective amount” refers to an amount of the compound (I) or a pharmaceutically acceptable salt thereof that will elicit the biological or medical response of a subject, or ameliorate symptoms, alleviate conditions, slow or delay disease progression, or prevent a disease, etc. In one embodiment, the term “a therapeutically effective amount” refers to the amount of the compound (I) or a pharmaceutically acceptable salt thereof that, when administered to a subject, is effective to (1) at least partially alleviating, inhibiting, preventing and/or ameliorating a condition, or a disorder or a disease (i) mediated by HMGB1 or (ii) associated with HMGB1 quantity, or (iii) characterized by activity of HMGB1; or (2) reducing the activity and/or quantity of HMGB1.

In practicing the invention, the effective amount of the compound (I) or a pharmaceutically acceptable salt thereof depends on a subject, age, sex, body weight, pathological condition and severity thereof, route of administration, and judgment of the prescribing subject. A determination of the amount based on such factors is within the competence of one of ordinary skill in the art, with the daily dose of the compound (I) or a pharmaceutically acceptable salt thereof being, for example, 0.001 to 1 mg per kg body weight per day, specifically 0.01 to 0.5 mg per kg body weight per day, more specifically 0.01 to 0.2 mg per kg body weight per day, more specifically 0.02 to 0.2 mg per kg body weight per day, but not limited to them.

In practicing the invention, the effective amount of the compound (I) or a pharmaceutically acceptable salt thereof is administered once to three times daily, but is not limited thereto.

In practicing the invention, the compound (I) or a pharmaceutically acceptable salt thereof may be used for the treatment of HMGB1-dependent diseases in combination with other active compounds, for example, with a peptide defined by general formula of U.S. Pat. No. 4,707,468

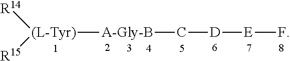

wherein R¹⁴ and R¹⁵ can be the same or different and each is hydrogen, lower alkyl or lower alkenyl; A is selected from the group consisting of D-Met, D-Ala, D-Ser, D-Cys, D-Thr, Gly and Sar, provided that when A is D-Cys, it is bonded with L-Cys or D-Cys located in position 5 through a S—S bond to effect intramolecular ring closure; B is L-Phe or D-Phe in which the benzene ring can be substituted or an α-N-alkyl derivative thereof; C is selected from the group consisting of L-Leu, L-Ile, L-Nle, L-tert.-Leu, L-Met, L-Met(O), L-Ser, L-Cys, L-Val, D-Cys and α-N-alkyl derivatives thereof, provided that when A is D-Cys, C is L-Cys or D-Cys and C is bonded with A through said S—S bond; D and E each is selected from the group consisting of L-Arg, D-Arg, L-Lys, D-Lys, L-homo-Arg, D-homo-Arg, L-Orn, D-Orn and α-N-alkyl derivatives thereof; F is OR³,

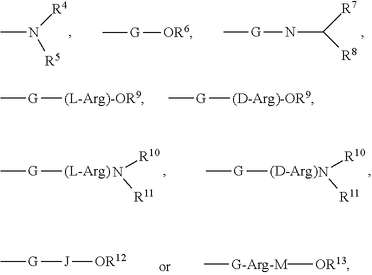

in which R³, R⁴, R⁵ R⁶, R⁷, R⁸, R⁹, R¹⁰, R¹¹, R¹², and R¹³ are. the same or different and each is hydrogen or lower alkyl, G is selected from the group consisting of Gly, Ala, Val, Leu, Ile, Ser, Thr, Cys, Met, Asp, Glu, Asn, Gln, Pro, Lys, Orn, Arg, His, Phe, Tyr, Trp, tert.-Leu, 2-aminoisobutyric acid, α-methyl-Leu, β-alanine, γ-aminobutyric acid and α-N-alkyl derivatives thereof, J is selected from the group consisting of Gly, Sar, L-Ala, D-Ala, L-Phe, D-Phe, L-Asp and D-Asp, and M is selected from the group consisting of D-Pro, D-ala and D-Glu, provided that at least one of A, B, C, D, E and F comprises a D-amino acid or an N-alkyl derivative of a D-amino acid or a L-amino acid, or a pharmacologically acceptable salt of said peptide. In view of findings of the present invention, it is expected that any peptide defined by general formula of U.S. Pat. No. 4,707,468 would necessarily have the anti-HMGB1 effect and be useful in the treatment of HMGB1-dependent diseases.

The compound (I) or a pharmaceutically acceptable salt thereof may be used to advantage in a combination with at least one other active pharmaceutical agent for simultaneous or sequential administration, e.g., in the treatment or prevention of rheumatoid arthritis, osteoarthritis, ankylosing spondylitis, periodontitis, allergic rhinitis, chronic rhinosinusitis without nasal polyps, chronic rhinosinusitis with nasal polyps, pneumonia, chronic obstructive pulmonary disease, asthma, acute adult respiratory syndrome, diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, diabetic foot ulceration, colitis, inflammatory bowel disease, and ocular disease. Such the pharmaceutical combination may be in a unit dosage form comprising a predetermined quantity of each of the at least two active components. Alternatively, the pharmaceutical combination may comprise the at least two active components separately. Non-exclusive examples of such at least one other active pharmaceutical agent include hydroxychloroquine, leflunomide, methotrexate, sulfasalazine, olsalazine, balsalazide, minocycline, abatacept, rituximab, tocilizumab, anakinra, adalimumab, etanercept, infliximab, certolizumab pegol, golimumab, tofacitinib, baricitinib, celecoxib, ibuprofen, nabumetone, naproxen sodium, naproxen, piroxicam, diclofenac, diflunisal, indomethacin, ketoprofen, etodolac, fenoprofen, flurbiprofen, ketorolac, meclofenamate, mefenamic acid, meloxicam, oxaprozin, sulindac, salsalate, tolmetin, betamethasone, prednisone, dexamethasone, cortisone, hydrocortisone, methylprednisolone, prednisolone, immunosup cyclophosphamide, cyclosporine, azathioprine, glycyrrhizic acid, hydroxychloroquine, triamcinolone acetonide, oxymetazoline, phenylephrine, pseudoephedrine, albuterol, levalbuterol, ipratropium, aclidinium, arformoterol, formoterol, glycopyrrolate, indacaterol, olodaterol, revefenacin, salmeterol, tiotropium, umeclidinium, beclomethasone, budesonide, flunisolide, fluticasone, mometasone, montelukast, zafirlukast, zileuton, insulin, regular insulin, insulin aspart, insulin glulisine, insulin lispro, insulin isophane, insulin degludec, insulin detemir, insulin glargine, insulin glargine, acarbose, miglitol, metformin, bromocriptine, dipeptidyl peptidase-4 inhibitors, albiglutide, dulaglutide, exenatide, liraglutide, semaglutide, nateglinide, repaglinide, sodium-glucose transporter (SGLT) 2 inhibitors, sulfonylureas, rosiglitazone, pioglitazone, mesalamine, tocacitinib, azathioprine, and mercaptopurine.

The following examples are presented to demonstrate the invention. The examples are illustrative only and are not intended to limit the scope of the invention in any way.

### EXAMPLE 1

This example illustrates the process for preparing compound (I).

Benzyl chloroformate (2.55 g; 15.0 mmol) was added by portions to 50 ml solution of commercially available H-Tyr-D-Ala-Gly-Phe-Leu-Arg-OH diacetate (10.0 g; 11.8 mmol; IV) in water adjusted to alkaline pH by addition of 15 ml of 3N NaOH and stirred overnight. Then, the mixture was acidified with acetic acid, evaporated in vacuum to dryness, and the dry residue was extracted with ethanol. Evaporating ethanol extract yields dry solid Cbz-Tyr-D-Ala-Gly-Phe-Leu-Arg-OH (V) acetate (10.1 g; 11.0 mmol). It was dissolved in dimethylacetamide and L-arginine methyl ester dihydrochloride (3.1 g; 12.0 mmol; Sigma-Aldrich 11030), dicyclohexylcarbodiimide (2.5 g; 12.0 mmol; DCC), and triethylamine (1.2 g; 12.0 mmol) were added to the solution. The resulting mixture was stirred for 4 hours at 0° C. and then evaporated to dryness in vacuum. The dry solid was exchanged to acetate on anion resin AG1-X8 (quaternary ammonium) by standard procedure to provide N-protected heptapeptide methyl ester (II) acetate as a white solid, yield about 65% based on the starting hexapeptide (IV). Cbz-Tyr-D-Ala-Gly-Phe-Leu-Arg-Arg-OCH₃ acetate (II): HRMS (ESI) m/z [M+H⁺] calcd. for C₅₀H₇₁N₁₃O₁₁: 1030.5400, found 1030.5409.

The methyl ester (II) was converted to desired amides by reacting of methyl ester (II) (1.0 g, 0.9 mmol) with ethylamine (90.2 mg, 2.0 mmol), butylamine (146.5 mg, 2.0 mmol), or dimethylamine (90.1 mg, 2.0 mmol) in 25 ml of methanol, or ammonia solution, prepared by saturation of 25 ml of methanol with dry ammonia gas. The solutions were stirred for two days to prepare Cbz-Tyr-D-Ala-Gly-Phe-Leu-Arg-Arg-NH₂, Cbz-Tyr-D-Ala-Gly-Phe-Leu-Arg-Arg-NHC₂H₅, Cbz-Tyr-D-Ala-Gly-Phe-Leu-Arg-Arg-NHC₄H₉, or Cbz-Tyr-D-Ala-Gly-Phe-Leu-Arg-Arg-N(CH₃)₂, respectively. After removing excess of amines/ammonia and solvent by evaporation in vacuum, the desired products were obtained as dry solids. Then, the products were dissolved in MeOH (20 ml) with acetic acid (0.2 g, 3.3 mmol) and the solution was flushed with H₂ stream in the presence of 10% palladium on charcoal at room temperature until the reaction was complete. The mixtures were filtered and filtrates were evaporated in vacuum until dryness. The dry residues were purified using preparative HPLC to provide desired amides as white solids with yields 63-81% based on the starting methyl ester (II).

H-Tyr-D-Ala-Gly-Phe-Leu-Arg-Arg-NH₂ acetate, yield 74%: ¹ NMR (400 MHz, D₂O), δ-scale: 0.80 (d, J=4 Hz, 3H), 0.85 (d, J=4 Hz, 3H), 1.09 (d, J=8 Hz, 3H), 1.40-1.83 (m, 11H), 1.87 (s, 9H), 2.89 (m, 1H), 2.95-3.09 (m, 3H), 3.14 (m, 4H), 3.80 (m, 3H), 4.09 (m, 1H), 4.25 (m, 3H), 4.54 (m, 1H), 6.83 (m, 2H), 7.07 (m, 2H), 7.19 (m, 2H), 7.28 (m, 3H). HRMS (ESI) m/z [M+H⁺] calcd. for C₄₁H₆₄N₁₄O₈: 881.5032, found 881.5039.

H-Tyr-D-Ala-Gly-Phe-Leu-Arg-Arg-NHC₂H₅ acetate, yield 81%: HRMS (ESI) m/z [M+H⁺] calcd. for C₄₃H₆₈N₁₄O₈: 909.5345, found 909.5351.

H-Tyr-D-Ala-Gly-Phe-Leu-Arg-Arg-NHC₄H₉ acetate, yield 63%: HRMS (ESI) m/z [M+H⁺] calcd. for C₄₅H₇₂N₁₄O₈: 937.5658, found 937.5651.

H-Tyr-D-Ala-Gly-Phe-Leu-Arg-Arg-N(CH₃)₂ acetate, yield 69%: HRMS (ESI) m/z [M+H⁺] calcd. for C₄₃H₆₈N₁₄O₈: 909.5345, found 909.5349.

### EXAMPLE 2

This example illustrates efficacy of compounds of the invention in the inhibition of HMGB1 secretion.

Compounds of the invention were tested in a mouse model of acute lung injury induced by intratracheal administration of lipopolysaccharide (LPS) as described, for instance, in D'Alessio F R. Mouse Models of Acute Lung Injury and ARDS. Methods Mol Biol. 2018, 1809:341-350. C57BI/6 male mice were randomized into eight groups of 6 animals each. Mice were treated intratracheally with 1 μg/mouse of α-Galactosyl Ceramide (α-GalCer, KRN7000, Sigma-Aldrich) and after 24 hours with LPS (E. coli; 1 mg/mouse) and 10 μL/mouse Freund's complete adjuvant. Then, mice received by inhalation 50 μL saline (control); 10, 100, or 250 μg/kg of H-Tyr-D-Ala-Gly-Phe-Leu-Arg-Arg-NH₂; 100 μg/kg of H-Tyr-D-Ala-Gly-Phe-Leu-Arg-Arg-NHC₂H₅; 100 μg/kg of H-Tyr-D-Ala-Gly-Phe-Leu-Arg-Arg-NHC₄H₉; 100 μg/kg of H-Tyr-D-Ala-Gly-Phe-Leu-Arg-Arg-N(CH₃)₂; 10 μg/kg H-Tyr-D-Ala-Gly-Phe-Leu-Arg-Arg-OH (Chavkin C et al. 1981); or 250 μg/kg of [D-Ala², D-Leu⁵]-enkephalin as solutions in 50 μL. saline. After 48 hours, HMGB1 concentrations were measured in bronchoalveolar fluid (BALF) with commercially available ELISA kit for HMGB1 (SEA3999Mu, Cloud-Clone corp.). Results are presented in Tables 1 as mean±SEM (n=6) of HMGB1 levels in BALF, in % of control.

TABLE 1

| Treatment, dose | HMGB1 in BALF, % |
|---|---|
| Control (saline) | 100.0 ± 9.5 |
| H-Tyr-D-Ala-Gly-Phe-Leu-Arg-Arg-NH₂ acetate, 10 μg/kg | 60.3 ± 9.0* |
| H-Tyr-D-Ala-Gly-Phe-Leu-Arg-Arg-NH₂ acetate, 100 | 31.8 ± 12.4* |
| μg/kg | |
| H-Tyr-D-Ala-Gly-Phe-Leu-Arg-Arg-NH₂ acetate, 250 | 34.2 ± 8.0* |
| μg/kg | |
| H-Tyr-D-Ala-Gly-Phe-Leu-Arg-Arg-NHC₂H₅ acetate, 100 | 35.2 ± 7.2* |
| μg/kg | |
| H-Tyr-D-Ala-Gly-Phe-Leu-Arg-Arg-NHC₄H₉ acetate, 100 | 40.1 ± 5.0* |
| μg/kg | |
| H-Tyr-D-Ala-Gly-Phe-Leu-Arg-Arg-N(CH₃)₂ acetate, 100 | 42.3 ± 5.9* |
| μg/kg | |
| H-Tyr-D-Ala-Gly-Phe-Leu-Arg-Arg-OH acetate, 10 μg/kg | 88.67 ± 10.5 |
| [D-Ala², D-Leu⁵]-enkephalin, 250 μg/kg | 92.3 ± 7.2 |

*Differs significantly of control (p < 0.05; one-way ANOVA, Tukey's post-test).

Table 1 shows that compounds of the invention significantly reduced HMGB1 secretion compared to control, thereby preventing a late-phase inflammation in the LPS-induced mice, while the structurally similar peptide [D-Ala², D-Leu⁵]-enkephalin at dose of 250 μg/kg was ineffective (p>0.05). The amide H-Tyr-D-Ala-Gly-Phe-Leu-Arg-Arg-NH₂ acetate (10 μg/kg) was significantly more effective than the structurally similar peptide H-Tyr-D-Ala-Gly-Phe-Leu-Arg-Arg-OH acetate, taken at the same dose (p>0.05). Lowering HMGB1 levels upon treatment with compounds of the present invention was accompanied with alleviating symptoms of respiratory disease such as disturbances in respiration, decrease in appetite, and inhibition of motor activity as compared to control. Thus, compounds of the present invention are useful in the treatment of the HMGB1-dependent inflammatory lung disease.

### EXAMPLE 3

This example illustrates that compound (I) is useful for treating arthritis, the HMGB1-dependent disease.

An efficacy of the compound of the invention was tested in a mouse model of rheumatoid arthritis, as described in Brand et al., Nat Protoc. 2007, 2(5):1269-75. Male BALB/c mice were randomized into two groups of 5 animals each. Arthritis was induced in using an intradermal injection of cold emulsion (0.05 ml) prepared by emulsifying chicken type II collagen (CII) solution in acetic acid with complete Freund's adjuvant (CFA) into the base of the tail. Three weeks later, the mice were administered by a second injection in the same manner. Following the booster immunization, mice received 50 μL saline i.p. (control) or 100 μg/kg H-Tyr-D-Ala-Gly-Phe-Leu-Arg-Arg-NH₂ in 50 μL saline i.p., once-a-day, for two weeks. The serum level of HMGB1 was determined on day 35 with commercially available ELISA kit for HMGB1 (SEA3999Mu, Cloud-Clone corp.). The clinical severity of arthritis in each paw was quantified according to a graded scale from 0 to 4, as described in Brand et al., Nat Protoc. 2007, 2(5):1269-75. Results are presented in Table 2 as and mean±SEM (n=5) of HMGB1 levels in serum in % of control and arthritis incidence.

TABLE 2

| | HMGB1 in | |
|---|---|---|
| Treatment | serum, % | Incidence, % |
| Control | 100.0 ± 23.3 | 80 |
| H-Tyr-D-Ala-Gly-Phe-Leu-Arg-Arg-NH₂ | 21.5 ± 5.9* | 20 |

*Differs significantly of control (p < 0.05; unpaired two-tailed t-test).

Table 2 shows that compound (I) significantly decreased HMGB1 secretion in mice with type II collagen-induced arthritis (p<0.05) and reduced arthritis incidence compared to control. Thus, compound (I) is useful in the treatment of HMGB1-dependent arthritis.

### EXAMPLE 4

This example illustrates an aqueous pharmaceutical composition for injections comprising the compound (I).

The aqueous pharmaceutical composition is prepared as follows. H-Tyr-D-Ala-Gly-Phe-Leu-Arg-Arg-NH₂ acetate is dissolved in water for injections in an amount as indicated in table 3 and the solution is adjusted to pH 3.0-8.5 using phosphate buffer, yield 100%.

TABLE 3

| Ingredient | Content, % w/w |
|---|---|
| H-Tyr-D-Ala-Gly-Phe-Leu-Arg-Arg-NH₂ acetate | 0.01-1.00 |
| Phosphate buffer | pH 3.0-8.5 |
| Water for injections | up to 100.00 |

The solution is filtered through a filter with a pore size of 0.22 microns (Merck Millipore) to prepare a sterile, preservative-free solution, yield 99.9%. The solution is filled into sterile ampules in a stream of nitrogen or any other oxygen-free inert gas and sealed. The obtained aqueous pharmaceutical composition in a unit dosage form can be administered to a subject in need thereof as injections or intravenous infusions for the treatment of a HMGB1-dependent inflammatory or autoimmune disease.

### EXAMPLE 5

This example illustrates an aqueous pharmaceutical composition for inhalation comprising the compound (I).

The aqueous pharmaceutical composition is prepared as follows. H-Tyr-D-Ala-Gly-Phe-Leu-Arg-Arg-NH₂ acetate is dissolved in water for injections in an amount as indicated in table 4, yield 100%.

TABLE 4

| Ingredient | Content, % w/w |
|---|---|
| H-Tyr-D-Ala-Gly-Phe-Leu-Arg-Arg-NH₂ acetate | 0.1-0.5 |
| Water for injections | up to 100.00 |

The solution is filtered through a filter with a pore size of 0.22 microns (Merck Millipore) to prepare a sterile, preservative-free solution, yield 99.9%. The solution is filled into sterile ampules in a stream of nitrogen or any other oxygen-free inert gas and sealed. The obtained aqueous pharmaceutical composition in a unit dosage form can be administered to a subject in need thereof as inhalations for the treatment of a HMGB1-dependent inflammatory or autoimmune lung disease.

### EXAMPLE 6

This example illustrates a solid pharmaceutical composition for rectal administration comprising the compound (I).

The solid pharmaceutical composition is prepared as follows. To prepare a suppository mass, 1788.9 g of Witepsol W35 and 100 g of cocoa butter are mixed and heated to 45° C. with stirring until completely melted, then the melt is cooled to 40° C. In a separate container, 11.1 g of H-Tyr-D-Ala-Gly-Phe-Leu-Arg-Arg-NH₂ acetate is dissolved in 100 g of purified water in the proportions indicated in Table 5 until a clear solution is formed. The prepared aqueous solution is poured onto the heated melt of Witepsol W35/cocoa butter and mixture is homogenized. At the end of homogenization, the mass is transferred to a separate container, yield is 98.5%. An empty suppository tape is placed in the holder of the filling machine and the temperature of the filling container for the suppository mass is set to 40° C. After filling, the suppositories are left to cool in the filled tape storage compartment. The stage yield is 99% (based on the basic substance). The frozen suppositories are fixed on the tape holder, the process of sealing and cutting the suppository tape is started. The finished product is a suppository weighing 2 g, with a compound(I) content of 10 mg. The composition of the suppositories is shown in Table 5.

TABLE 5

| Ingredient | Content, % w/w |
|---|---|
| H-Tyr-D-Ala-Gly-Phe-Leu-Arg-Arg-NH₂ acetate | 0.5 |
| Purified water | 5.0 |
| Cacao butter | 5.0 |
| Witepsol W35 | up to 100 |

The obtained suppositories can be administered to a subject in need thereof rectally for the treatment of a HMGB1-dependent inflammatory or autoimmune disease.

### EXAMPLE 7

This example illustrates that compound (I) is useful for treating arthritis, the HMGB1-dependent disease.

An efficacy of H-Tyr-D-Ala-Gly-Phe-Leu-Arg-Arg-NH₂ acetate was tested in a mouse model of antigen-induced arthritis induced by methylated bovine serum albumin (mBSA), as described in Brackertz et al., Arthritis Rheum. 1977, 20(3):841-50; Staite et al., Agents and Actions 1989, 27(3/4):338-40. Briefly, male C3H/He mice 8-10 weeks of age were randomized into two groups of 6 animals each. Immunization: the mice were immunized 2 times (on the 0^{th} and 7^{th} days) with s.c. injections of mBSA (50 mg/kg, Sigma-Aldrich) emulsified in 20 μL Freund's complete adjuvant and on the 21st day received intra-articular injection of mBSA (50 mg/kg) in 20 μL saline in the right hind knee as a booster immunization. It resulted in the knee swelling, with a 1.7-fold increase in the knee joint circumference from 1.20±0.03 to 2.03±0.07 cm (p<0.0001) on the 25th day. Treatment: the mice received s.c. injections either 0.1 ml saline (control, n=6) or 0.14 mg/kg H-Tyr-D-Ala-Gly-Phe-Leu-Arg-Arg-NH₂ acetate in 0.1 ml saline (n=6), once-a-day for 5 consecutive days, beginning 30 min after the booster immunization. The disease course was quantified by assessment of the swelling of the arthritic knee joint on 25th day. HMGB1 levels (pg/ml) in the synovial lavage fluids of the arthritic joint on 25th day was measured using ELISA (SEA3999Mu, Cloud-Clone corp.). Results are presented in tables 6 and 7 as mean±SEM arthritic knee joints circumference and HMGB1 levels in synovial lavage fluids, respectively.

TABLE 6

| | Knee joint |
|---|---|
| Group | circumference, cm |
| mBSA + Saline (control) | 2.03 ± 0.07 |
| mBSA + H-Tyr-D-Ala-Gly-Phe-Leu-Arg-Arg-NH₂ | 1.32 ± 0.04**** |
| acetate | |

****Differs significantly of control (p < 0.0001; two-tailed unpaired t-test).

TABLE 7

| Treatment | HMGB1, pg/ml |
|---|---|
| mBSA + Saline (control) | 248 ± 40 |
| mBSA + H-Tyr-D-Ala-Gly-Phe-Leu-Arg-Arg-NH₂ | 119 ± 38* |
| acetate | |

*Differs significantly of control (p < 0.05; two-tailed unpaired t-test).

Tables 6 and 7 show that compound (I) significantly reduced the knee swelling and elevated HMGB1 levels in mice with antigen-induced arthritis. Thus, compounds of the present invention are useful in the treatment of HMGB1-dependent arthritis.

1. A compound of formula (I):
H-Tyr-D-Ala-Gly-Phe-Leu-Arg-Arg-Z (I)
where Z is 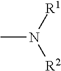
in which R¹ and R² are identical or different and are hydrogen or a lower alkyl chain having up to 4 carbon atoms or a pharmaceutically acceptable salt thereof.

2. The compound according to claim 1, wherein said compound is selected from the group consisting of H-Tyr-D-Ala-Gly-Phe-Leu-Arg-Arg-NH₂ acetate, H-Tyr-D-Ala-Gly-Phe-Leu-Arg-Arg-NHC₂H₅ acetate, H-Tyr-D-Ala-Gly-Phe-Leu-Arg-Arg-NHC₄H₉ acetate, and H-Tyr-D-Ala-Gly-Phe-Leu-Arg-Arg-N(CH₃)₂ acetate.

3. The compound according to claim 1, wherein said compound is H-Tyr-D-Ala-Gly-Phe-Leu-Arg-Arg-NH₂ acetate.

4. A pharmaceutical composition comprising the compound according to claim 1 and a pharmaceutically acceptable excipient.

5. A method of administering a medicament comprising the compound according to claim 1.

6. A method comprising the step of administering the compound according to claim 1 in the treatment of a HMGB1-dependent inflammatory or autoimmune disease selected from the group consisting of rheumatoid arthritis, osteoarthritis, ankylosing spondylitis, periodontitis, allergic rhinitis, chronic rhinosinusitis without nasal polyps, chronic rhino sinusitis with nasal polyps, pneumonia, chronic obstructive pulmonary disease, asthma, acute adult respiratory syndrome, diabetic macroangiopathy, diabetic coronary artery disease, peripheral arterial disease, diabetic cerebrovascular disease, diabetic microangiopathy, diabetic nephropathy, diabetic neuropathy, diabetic retinopathy, diabetic cardiomyopathy, diabetic foot ulceration, colitis, inflammatory bowel disease, and ocular disease.

7. The compound according to claim 1, wherein the pharmaceutically acceptable salt is acetate.
